(19) United States
(12) Patent Application Publication
(10) Pub. No.: US 2018/0179502 A1
(43) Pub. Date: Jun. 28, 2018

(54) Nucleic Acid Analogue-Guided Chemical Nuclease System, Methods and Compositions

(71) Applicant: Chen-Yu Liu, Middletown, DE (US)

(72) Inventor: Chen-Yu Liu, Middletown, DE (US)

(73) Assignee: Genalyze LLC, Middletown, DE (US)

(21) Appl. No.: 15/831,381

(22) Filed: Dec. 4, 2017

(30) Foreign Application Priority Data
Dec. 2, 2016 (US) 6242971

(51) Int. Cl. C12N 9/22; C07K 14/00

(52) U.S. Cl. CPC C12N 9/22; A61P 31/04; C07K 14/003

(57) ABSTRACT

Nucleic acid analogue-guided chemical nuclease systems, methods and compositions, which can manipulate the genome DNA sequence in a sequence-specific manner. The core components that together make up the architecture of the system are: nucleic acid analogues (e.g. Peptide nucleic acids) and bleomycin or its derivatives. Generally speaking, these components are structured such that nucleic acid analogues which recognize specific DNA sequences are conjugated ( covalently) to bleomycin or its derivatives which can cleave the target DNA. This architecture allows the method to sequence-specifically insert or remove DNA sequence from the target DNAs.

### CROSS-REFERENCE TO RELATED APPLICATIONS

This application claims priority from U.S. Provisional Application 62/429,714 entitled “Nucleic Acid Analogue-Guided Chemical Nuclease Systems, Methods and Compositions”, filed Dec. 2, 2016, U.S., and herein incorporated by reference in its entirety.

### TECHNICAL FIELD OF THE INVENTION

The present invention generally relates to systems, methods and compositions used for the control of gene expression with DNA cleavage involving sequence targeting, such as genome editing, that may use nucleic acid analogues and components thereof.

### BACKGROUND OF THE INVENTION

Most of biological functions of a cell depend on its gene expression profiles. Genome editing technologies have been provided as means for altering gene expression profiles of a cell by insertion or deletion of target DNA sequences that encode gene or regulate gene expression. Therefore, genome editing technologies completely remove or add DNA sequences in the target sites and turn off a gene expression or add a new gene expression in the gene expression profiles. The targeting nuclease to target sites can become potent biomedical tools.

Although, during the past several decades, it has a great progress in the technologies of preparing and engineering sequence-specific DNA binding proteins, their applications in genome editing to the regulation of gene expression are still limited by their expensive selection process (e.g., Pingoud et al., ChemBioChem 12, 1495-1500 (2011)).

Or in other genome editing approach-Clustered Regularly-interspaced Short Palindromic Repeats (CRISPR) that relies on nucleic acid- nucleic acid interactions to target its modification sites. This type of approach also has the difficulty of use in vivo for delivery since its delivery into cell is via either viral transfection or encapsulating the bulk system with cationic lipid (Timin et al., Nanomedicine. pii: S1549-9634 (17)30168-5 (2017)).

Or in another approach, the peptide nucleic acids were bound to the target genome DNA sequence and triggered nucleotide excision repair (NER) pathway and activate the target site for DNA recombination. With the nucleic acids encoding modified sequences introduced into a cell with the peptide nucleic acid, the modified nucleic acids can be incorporated into the target site. However, the nucleotide excision repair mechanism is not as efficient as directly using nuclease to cleave the DNA target site in order to trigger the homogeneous recombination (HR) of DNA (Schleifman et al., Chem Biol.,18(9): 1189-1198 (2011)).

This invention discloses a method and system, that uses nucleic acid analogues as a DNA binding domain. That is easy to set up and affordable.

### SUMMARY OF THE INVENTION

Disclosed herein are compositions and methods useful for genome editing at predetermined sites. These compositions and methods are useful for deletion or insertion of DNA sequences on cellular DNA at predetermined sites. The techniques disclosed herein may be used as laboratory tools to study gene expression, and/or as methods of treatment for diseases and disorders associated with gene or protein expression. The techniques disclosed herein can also be used as a novel type of antibiotics which kills bacteria with species specificity or an agent to remove viral DNA from the host.

The present invention advantageously fills the aforementioned deficiencies by providing nucleic acid analogue-guided chemical nuclease which provides a mean for insertion or deletion of predetermined DNA sequences of cellular DNA.

The present invention is a nucleic acid analogue-guided genome editing method. In some embodiments, the method comprises artificial chimeric molecules which comprise of nucleic acids analogues and bleomycin or its derivatives. The nucleic acids analogues and bleomycin or its derivatives are conjugated covalently.

In some embodiments, the nucleic acids analogues are able to recognize and bind specifically to the target DNA sequences of cellular DNA, and the bleomycin or its derivatives can cleave the polynucleotide on the target sites, which may allow modification of the target DNA sequence by non-homogenous end join pathway (NHEJ) or insertion of the target DNA by homogeneous recombination (HR) pathway with remedy DNA fragments whereby it allows the insertion or deletion of predetermine DNA sequence on the target sites. Wherein, the nucleic acids analogues of artificial chimeric molecules are non-naturally occurring.

In some embodiments, the nucleic acids analogues are able to recognize and bind specifically to the target DNA sequences of bacteria or viral DNA, and the bleomycin or its derivatives can cleave the polynucleotide on the target sites. Said the target sites may be encoded with essential DNA sequences for bacterial or virus to replicate.

The present invention is unique when compared with other known solutions because it does not require the generation of customized proteins to target specific DNA sequences but rather nucleic acid analogues to recognize specific DNA targets, in other words, the bleomycin or its derivatives can be recruited to a specific DNA target using said nucleic acid analogues. In addition, the bleomycin or its derivatives are low immunogenic and have been used as a medication for decades. Also, a plurality of nucleic acid analogues binding modes provide more options for the choices of DNA target sites. In addition, putting together the nucleic acid analogues guided genome editing method with the genome sequencing techniques and analysis methods, it may significantly simplify the methodology and enhance the ability to clarify the association among genes and a diverse range of biological functions and diseases. The present invention is unique and structurally different from other methods. More specifically, the present invention is unique due to the presence of nucleic acids analogues, which is different with any other genome editing method that is based on protein-protein chimeras; and the nucleic acids analogues also provide various binding modes which distinguish the invention from current all other solutions. Also, the invention contains the non-natural occurring nucleic acids analogues which does not need the whole artificial chimeric molecules delivered into cells through viral infection. Further more, the artificial chimeric molecules are resistant to degradation in cells. Further more, the bleomycin or its derivatives as DNA cleavage reagent for activation of homogeneous recombination (HR) and non-homogenous end join (NHEJ) at DNA sequence specific manner.

In certain embodiments, the disclosure provides the bleomycin or its derivatives of an artificial chimeric molecule comprising the activity of nuclease.

In one embodiment, the disclosure provides a method for modifying a region of interest in cellular chromatin, wherein the method comprises contacting cellular DNA with an artificial chimeric molecule that binds to a binding site in the region of interest (Examples of an interest region are promoter/encoding regions of genes listed in Tables A,B,C of Appendix A—which is derived from U.S. Pat. No. 8,697,359, issued Apr. 15, 2014, and entitled “CRISPR-Cas systems and methods for altering expression of gene products”).

The artificial chimeric molecule comprises: 1) a DNA binding domain, and 2) a component of bleomycin or its derivatives thereof.

In one embodiment, the disclosure provides a method for cleaving a region of interest in bacterial chromosome, wherein the method comprises contacting bacterial chromosome DNA with an artificial chimeric molecule that binds to a binding site in the region of interest within bacterial chromosome. The artificial chimeric molecule comprises: 1) a DNA binding domain, and 2) a component of bleomycin or its derivatives thereof.

In one embodiment, the disclosure provides a method for cleaving a region of interest in viral DNA, wherein the method comprises contacting viral DNA with an artificial chimeric molecule that binds to a binding site in the region of interest within viral DNA. The artificial chimeric molecule comprises: 1) a DNA binding domain, and 2) a component of bleomycin or its derivatives thereof.

In a more preferred embodiment, an artificial chimeric molecule is a fusion polypeptide comprising a peptide nucleic acid (PNA) and bleomycin or its derivatives.

In some embodiments, an artificial chimeric molecule comprises of nucleic acids analogues and bleomycin or its derivatives. The nucleic acids analogues and bleomycin or its derivatives are conjugated covalently in a site specific manner.

In some embodiments, the nucleic acids analogues of the artificial chimeric molecules are able to recognize and bind specifically to the target DNA sequences, and the bleomycin or its derivatives can cleave the target DNAs. Wherein, the nucleic acids analogues of artificial chimeric molecules are non-naturally occurring.

In some embodiments, the target DNAs of the artificial chimeric molecules can can cleave the target DNAs to allow insertion of polynucleotide or deletion of a DNA sequence.

In one embodiment, the DNA binding domain comprises a nucleic acid analogue. In a more preferred embodiment, an artificial chimeric molecule is a polypeptide comprising a peptide nucleic acid. Other nucleic acid analogues for DNA-binding domains are also useful.

In one embodiment, this disclosure provides a method for modifying a region of interest in cellular DNA, wherein the method comprises contacting cellular DNA with an artificial chimeric molecule that binds the region of interest. The artificial chimeric molecule comprises a DNA binding domain and bleomycin or its derivatives with nuclease activity thereof. (Examples of an interest region are promoter/encoding regions of genes listed in Tables A,B,C of Appendix A—which is derived from U.S. Pat. No. 8,697,359, issued Apr. 15, 2014, and entitled “CRISPR-Cas systems and methods for altering expression of gene products”)

In one embodiment, cellular DNA can be present in prokaryotic, eukaryotic, archaeal cells.

In some embodiments, DNA binding domain comprises a peptide nucleic acid, the peptide nucleic acid binds to target DNA in a certain binding mode, or in a combination of the binding modes. These binding modes include but not limited to 1) peptide nucleic acid-double stranded DNA triplexes; and/or (2) triplex invasion complex; and/or (3) double duplex peptide nucleic acid invasion complexes; and/or (4) double stranded DNA/bis-peptide nucleic acid complexes; (5) single stranded DNA/peptide nucleic acid complexes (6) and/or strand invasion complexes at binding of γ-PNA.

In a more preferred embodiment, an artificial chimeric molecule contains tail-clamp peptide nucleic acid (tcPNA).

Genome editing is targeting of bleomycin or its derivatives to predetermined DNA sequences (target DNA sites), cleavaging on the predetermined DNA sequences in the proximity of predetermined DNA sequences.

### Method of Modifying Cellular DNA

In one embodiment, a target site in cellular DNA comprises a gene. The DNA in the proximity of exemplary genes can be modified via the method or composition disclosed herein. (The exemplary genes are listed in Table A, B, C of Appendix A—which is derived from U.S. Pat. No. 8,697,359, issued Apr. 15, 2014, and entitled “CRISPR-Cas systems and methods for altering expression of gene products”, which is incorporated herein.).

In one embodiment, cellular DNA contacts with a plurality of artificial chimeric molecules. Each artificial chimeric molecules target at distinct sites in a gene for cleavage of target DNA.

In one embodiment, an artificial chimeric molecule contains plurality of bleomycin or its derivatives domains. Each bleomycin or its derivatives domain may cleave a different site of the target DNA.

In one embodiment, cellular DNA contacts with a plurality of artificial chimeric molecules. The artificial chimeric molecules assemble together at target sites and cleave the target DNA.

In some embodiments, the disclosure provides for a method for creating a cell comprising of steps: introducing into a cell with artificial chimeric molecules. The artificial chimeric molecules bind to the DNA target sites and cleave the target DNA and allows insertion of DNA with donor polynucleotides. Propagation of cell produces the cell carrying the DNA with the inserted DNA, and determining an origin of the cell with the DNA sequence.

In some embodiments, the cell is selected from the cells list in table A of Appendix B, and transgenic varieties of these cells or any combination thereof. The available sources of cells are known to those with skill in the art (e.g., the American Type Culture Collection (ATCC)) Table A of Appendix B is derived from U.S. Pat. No. 8,697,359, issued Apr. 15, 2014, and entitled “CRISPR-Cas systems and methods for altering expression of gene products”).

In some embodiments, the propagation of cells produces a population of cells. In some embodiments, the organism is selected from the group comprising of: an animal or a plant. In some embodiments, the method further comprises cell selection. In some embodiments, the donor polynucleotide is inserted into a target DNA that is expressed in one cell. In some embodiments, the DNA with the insertion determines an origin of the cell. In some embodiments, the method comprises transplanting the cell into an organism.

In some embodiments, the disclosure provides a method for creating a cell line comprising of steps: introducing into a cell with artificial chimeric molecules. The artificial chimeric molecules bind to the DNA target sites and cleave the target DNA. Propagation of cells produces the cell line.

In some embodiments, after cleavage of target DNA, a donor polynucleotide is inserted into the target site.

In some embodiments, the disclosure also provides a method for insertion of donor polynucleotides into the target site.

In some embodiments, cell culturing may occur at any stage ex vivo. The cell or cells may even be re-introduced into the non-human animal or plant (including micro-algae).

In some embodiments, the method comprises collecting a cell or cells from a human or non-human animal or plant, and modifying the cell or cells. The cell or cells may even be re-introduced into the human or non-human animal or plant (including micro-algae).

### BRIEF DESCRIPTION OF THE DRAWING

A more complete understanding of the present invention may be had by reference to the following Detailed Description and appended claims when taken in conjunction with the accompanying Drawings.

FIG. 1 shows an example: a γ-peptide nucleic acid (PNA)-a nucleic acids analogue and its target DNA, in a sequence-specific manner, forms γ-PNA:DNA 1:1 strand-displacement duplex. The γ-PNA binds to a bleomycin (or its derivatives) via a site-specific cross-linking. The bleomycin (or its derivatives) may cleave the double-stranded, which is bound by the γ-PNA.

FIG. 2. shows an example: two PNA oligomers contained a part of identical (in a reverse manner) polypurine sequences joined by a flexible hairpin linker (called tail-clamp PNAs or tcPNA) stably binds to a double-stranded DNA (in a sequence -specific manner), forming a hybridization bubble. The tail-clamp PNAs is conjugated with bleomycin (or its derivatives). One of single-stranded DNA binds to the tail-clamp PNAs, and the other single-stranded DNA is displaced. The bleomycin (or its derivatives) on the tail-clamp PNAs can make a cleavage on the double-stranded DNA.

### DETAILED DESCRIPTION OF EXEMPLARY EMBODIMENTS

### Glossary of Terms:

The term “nucleic acid analogues” and “oligomers of nucleic acid” are used interchangeable herein, and they refer to oligomers of nucleic acid analogues which bind specifically to the predetermined DNA sequences. The non-limiting examples of nucleic acid analogues are peptide nucleic acid (PNA); γ-peptide nucleic acid; morpholino; locked nucleic acid (LNA). In some embodiments, the oligomers contain one or more other residues that do not belong to nucleic acid analogues.

The term “non-naturally occurring” indicates the involvement of the hand of man. The terms, when referring to artificial chimeric molecules mean that the nucleic acid analogues or their conjugations to polypeptide are not associated in nature and or found in nature.

The term “target DNA” is a predetermined DNA sequence.

The term “cellular DNA” refers to any DNA existing inside a cell.

The term “artificial chimeric molecule” used herein refers to a chimeric molecule with the activity of a nuclease and the specific DNA sequence binding ability.

The nucleic acid analogues and bleomycin or its derivatives domains are conjugated covalently. In some embodiments, the conjugation is through adding chemical functional groups on both nucleic acid analogues and bleomycin or its derivatives domains, and using chemical reagents or chemical crosslinkers to connect the nucleic acid analogues and bleomycin or its derivatives domains or polypeptides through the chemical functional groups in a site-specific manner. The non-limiting examples of covalent conjugation are crosslinking formation between the thiol groups on the cysteine residue of nucleic acid analogues and the primary amine of bleomycin or its derivatives domains, chemical crosslinkers connect nucleic acid analogues and bleomycin or its derivatives domains (see, for example, Greg T. Hermanson, Bioconjugate Techniques, Third Edition).

The practice of the present invention uses, unless otherwise indicated, conventional techniques of chemistry, biochemistry, microbiology, cell biology, molecular biology, and genomics, which are known to those skill of the art. (CURRENT PROTOCOLS IN MOLECULAR BIOLOGY (Wiley); the series METHODS IN ENZYMOLOGY (Academic Press, Inc.), Molecular Cloning: A Laboratory Manual (Cold Spring Harbor Lab Press))

In one embodiment, the invention provides for methods of genome editing in a eukaryotic cell.

In one embodiment, the invention provides for methods of genome editing in a prokaryotic cell.

In one embodiment, the invention provides for methods of genome editing a viral genome inside a cell.

### Delivery Method:

The artificial chimeric molecules of this disclosure, a donor polynucleotide, a reporter element, a genetic element of interest, a component of a split system and/or any nucleic acid or proteinaceous molecule, which is necessary to embody the methods of this disclosure, can be packed with compartments for delivery to cells. The non-limiting examples for the delivery method include but not limited to lipofection, polyethyleneimine (PEI)-mediated transfection, nucleofection, microinjection, calcium phosphate precipitation, liposomes, calcium phosphate precipitation, nanoparticle-mediated nucleic acid delivery, immunoliposomes, fusion, polycation or lipid:nucleic acid conjugates, protoplastcell-penetrating peptide conjugates of nucleic acids analogues, nanoparticle, electroporation particle gun technology, DEAE-dextran mediated transfection. The system can be delivered to cells or target tissues.

Lipofection method can be found in (e.g., U.S. Pat. No. 5,049,386) and are commercially available (e.g., Transfectam™ and Lipofectin™M). The delivery methods by using cationic and neutral lipids can be found in (e.g., Chan et al., J Gene Med. Mar; 16(0): 84-96 (2014)). Method of peptide nucleic acid delivery into cell (e.g., Peptide Nucleic Acids, Methods and Protocols, Second Edition Edited by Peter E. Nielsen, Daniel H. Appella). Method of delivery of immunolipid complexes, (e.g., Crystal, Science 270:404-410 (1995); Gao et al., Gene Therapy 2:710-722 (1995); Ahmad et al., U.S. Pat. No. 4,946,787).

In some embodiments, an artificial chimeric molecules complexed with a nucleic acid is delivered to a cell.

### Kits

The disclosure provides a kit comprising at least one of: artificial chimeric molecule described herein or/and donor polynucleotides described herein.

In some embodiments, artificial chimeric molecules described herein are used to produce a transgenic animal or plant.

In some embodiments, the organism include but limited to livestock, crops, pulses and tubers, poultry, edible insects and algae. The transgenic animal or plant may be useful in producing a disease model or food. Transgenic algae or other plants may be useful in producing oil and biofuels.

The ability to use artificial chimeric molecules to carry out genome editing improve production and enhance traits of a plant or animal.

### Two Illustrated Embodiments

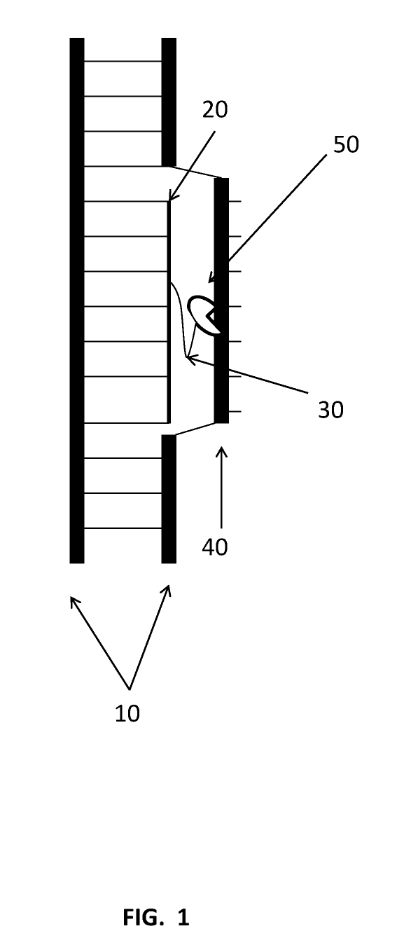

FIG. 1 depicts an exemplary embodiment of the method of the disclosure, a y peptide nucleic acid 20 is linked to a bleomycin (or its derivatives) 50 via a linker molecule 30. The whole construct is called an artificial chimeric molecule that can cleave the DNA 40 that is displaced by a y peptide nucleic acid 20 from a double stranded DNA 10. The linker molecule can be a crosslinker-SMCC for a covalent binding.

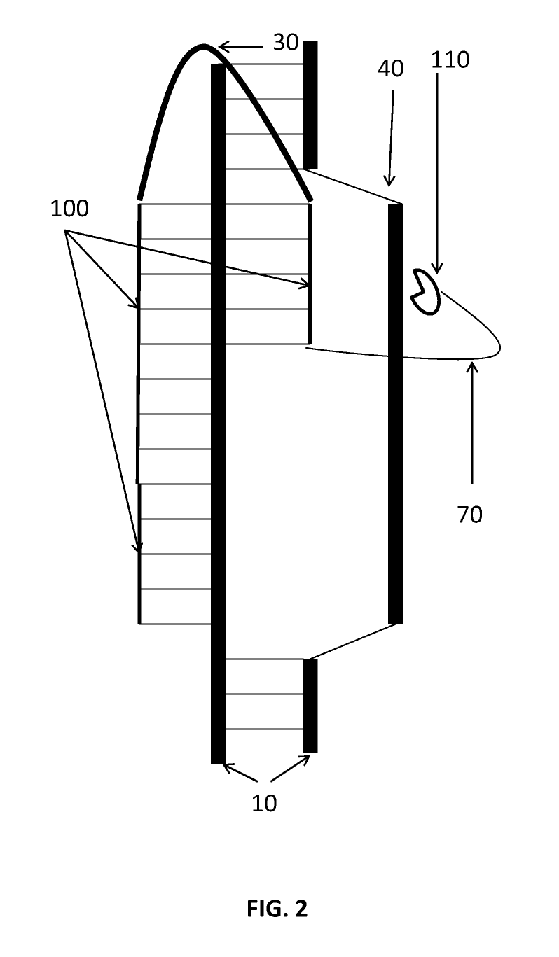

FIG. 2 depicts an exemplary embodiment of the method of the disclosure, a peptide nucleic acids 100 is linked to a bleomycin (or its derivatives) 110 via a linker molecule 70. The peptide nucleic acids 100 displaces the single-stranded DNA 40. The whole construct is called an artificial chimeric molecule system that can cleave the target double-stranded DNA 10.

### Exemplifications

The following examples are given for the purpose of illustrating various embodiments of the present disclosure and they are not meant to limit the disclosure in any fashion. These examples, along with the methods described herein are presently representative of preferred embodiments, are exemplary, and are not intended as limitations on the scope of the invention. Changes therein and other uses which are encompassed within the spirit of the disclosure as defined by the scope of the claims will occur to those skilled in the art.

### Example 1: Preparation of the Artificial Chimeric Molecule System

Trans-Cyclooctene-PEG4-NHS ester 0.5 mg was dissolved at 5 μl Dimethyl sulfoxide (DMSO). The bleomycin A5 1.48 mg was dissolved at 19 μl 50 mM phosphate buffer at pH 7.5. The conjugation of Trans-Cyclooctene-PEG4-NHS and bleomycin A5 was done by mixing the two solutions at room temperature for 3 hours. The product was dialyzed against a membrane with the molecule weight cutoff at 1 kDa. The product was trans-cyclooctene-PEG4 labeled Bleomycin A5. A peptid nucleic acid with a sequence:azido-AEEA-Lys-Lys-Lys-JTJTTJTTJT-AEEA-AEEA-AEEA-TCTTCTTCTCATTTC-Lys-Lys-Lys (10 nmol) dissolved at 100 μl 0.5M phosphate buffer at pH 7.5 was conjugated to Tetrazine-PEGS-NHS ester (0.5 mg) via its Lysine residues dissolved at corresponding amount of DMSO. The product was a mixture of peptide nucleic acids labeled with a various number of Tetrazine-PEGS from its lysine residues. The purification of product was through a desalt spin column which can remove molecules with molecular weight <7 kDa. The purified Trans-Cyclooctene-PEG4 labeled bleomycin A5 and the purified Tetrazine-PEGS labeled peptide nucleic acid was mixed at room temperature for 3 hours, and then the product was purified via desalt spin column with a molecular weight cut off <7 kDa. The final product was a mixture of peptide nucleic acids with various numbers of bleomycin conjugate.

### Example 2 Cleavage of Human CCR5 Sequence DNA with Artificial Chimeric Molecule System

A mixture of 20 μL 26 μM bleomycin A5 -tcPNA conjugate in 500 mM phosphate buffer at pH 7.5, 1 μL 0.15 mM (NH₄)₂Fe(SO₄)₂.6H₂O, 0.216M ascorbic acid, and 100 μL 100 μM oligonucleotide with human CCR5 sequence were added, and the incubation was continued for 1 hour at 37° C.

### Example 3: Genome Editing with the Artificial Chimeric Molecule System Illustrated in FIG. 1 and FIG. 2

In some embodiments, a artificial chimeric molecule is transfected into a cell.

In some embodiment, donor polynucleotides without the target DNA sequences are also introduced into the cell. The homologous recombination (HR) mechanism of a cell allows insertion of the donor nucleotides into the target DNA sites.

In some embodiments, the donor polynucleotides are not provided, the non-homologous end joining (NHEJ) mechanism of a cell mutates the DNA sequence of target sites.

### Example 4: Genome Editing

In some embodiments, a multiplexed artificial chimeric molecule is then introduced.

In some embodiments, one or more donor polynucleotides are introduced into the cell and the target nucleic acids are cleavaged by the artificial chimeric molecules. In some instances, the same donor DNA modified polynucleotides are incorporated into multiple cleavage sites.

### Example 5: Antivirus Drug

In some embodiments, one or more donor polynucleotides are introduced into the cell and the target nucleic acids are cleavaged by the artificial chimeric molecules. In some instances, the same donor DNA modified polynucleotides are incorporated into multiple cleavage sites.

### Example 6: Antibiotics

In some embodiments, one or more donor polynucleotides are introduced into the cell and the target nucleic acids are cleavaged by the artificial chimeric molecules. In some instances, the same donor DNA modified polynucleotides are incorporated into multiple cleavage sites.

All patents, patent applications and publications mentioned herein are hereby incorporated by reference in their entirety. Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### Appendix B

TABLE A
List of cell lines

C8161, CCRF-CEM, MOLT, mIMCD-3, NHDF, HeLa-S3, Huh1, Huh4, Huh7, HUVEC, HASMC, HEKn, HEKa,
MiaPaCell, Panel, PC-3, TF1, CTLL-2, C1R, Rat6, CV1, RPTE, A10, T24, J82, A375, ARH-77, Calu1, SW480,
SW620, SKOV3, SK-UT, CaCo2, P388D1, SEM-K2, WEHI-231, HB56, TIB55, Jurkat, J45.01, LRMB, Bcl-1, BC-
3, IC21, DLD2, Raw264.7, NRK, NRK-52E, MRC5, MEF, Hep G2, HeLa B, HeLa T4, COS, COS-1, COS-6, COS-
M6A, BS-C-1 monkey kidney epithelial, BALB/3T3 mouse embryo fibroblast, 3T3 Swiss, 3T3-L1, 132-d5
human fetal fibroblasts; 10.1 mouse fibroblasts, 293-T, 3T3, 721, 9L, A2780, A2780ADR, A2780cis, A172,
A20, A253, A431, A-549, ALC, B16, B35, BCP-1 cells, BEAS-2B, bEnd.3, BHK-21, BR 293, BxPC3, C3H-
10T1/2, C6/36, Cal-27, CHO, CHO-7, CHO-IR, CHO-K1, CHO-K2, CHO-T, CHO Dhfr-/-, COR-L23, COR-
L23/CPR, COR-L23/5010, COR-L23/R23, COS-7, COV-434, CMLT1, CMT, CT26, D17, DH82, DU145, DuCaP,
EL4, EM2, EM3, EMT6/AR1, EMT6/AR10.0, FM3, H1299, H69, HB54, HB55, HCA2, HEK-293, HeLa,
Hepa1c1c7, HL-60, HMEC, HT-29, Jurkat, JY cells, K562 cells, Ku812, KCL22, KG1, KYO1, LNCap, Ma-Mel
1-48, MC-38, MCF-7, MCF-10A, MDA-MB-231, MDA-MB-468, MDA-MB-435, MDCK II, MDCK II,
MOR/0.2R, MONO-MAC 6, MTD-1A, MyEnd, NCI-H69/CPR, NCI-H69/LX10, NCI-H69/LX20, NCI-H69/LX4,
NIH-3T3, NALM-1, NW-145, OPCN/OPCT cell lines, Peer, PNT-1A/PNT 2, RenCa, RIN-5F, RMA/RMAS,
Saos-2 cells, Sf-9, SkBr3, T2, T-47D, T84, THP1 cell line, U373, U87, U937, VCaP, Vero cells, WM39, WT-
49, X63, YAC-1, YAR

### Appendix A

TABLE A

| DISEASE/ | |
|---|---|
| DISORDERS | GENE(S) |
| Neoplasia | PTEN; ATM; ATR; EGFR; ERBB2; ERBB3; |
| | ERBB4; Notch1; Notch2; Notch3; Notch4; AKT; |
| | AKT2; AKT3; HIF; HIF1a; HIF3a; Met; HRG; Bcl2; |
| | PPAR alpha; PPAR gamma; WT1 (Wilms Tumor); |
| | FGF Receptor Family members (5 members: 1, 2, 3, 4, |
| | 5); CDKN2a; APC; RB (retinoblastoma); MEN1; |
| | VHL; BRCA1; BRCA2; AR (Androgen Receptor); |
| | TSG101; IGF; IGF Receptor; Igf1 (4 variants); Igf2 (3 |
| | variants); Igf 1 Receptor; Igf 2 Receptor; Bax; Bcl2; |
| | caspases family (9 members: 1, 2, 3, 4, 6, 7, 8, 9, 12); |
| | Kras; Apc |
| Age-related | Aber; Ccl2; Cc2; cp (ceruloplasmin); Timp3; |
| Macular | cathepsinD; Vldlr; Ccr2 |
| Degeneration | |
| Schizophrenia | Neuregulin1 (Nrg1); Erb4 (receptor for Neuregulin); |
| | Complexin1 (Cplx1); Tph1 Tryptophan hydroxylase; |
| | Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; |
| | DAOA; DTNBP1; Dao (Dao1) |
| Trinucleotide | HTT (Huntington's Dx); SBMA/SMAX1/AR |
| Repeat | (Kennedy's Dx); FXN/X25 (Friedrich's Ataxia); ATX3 |
| Disorders | (Machado-Joseph's Dx); ATXN1 and ATXN2 |
| | (spinocerebellar ataxias); DMPK (myotonic |
| | dystrophy); Atrophin-1 and Atn 1 (DRPLA Dx); CBP |
| | (Creb-BP-global instability); VLDLR (Alzheimer's); |
| | Atxn7; Atxn10 |
| Fragile X | FMR2; FXR1; FXR2; mGLUR5 |
| Syndrome | |
| Secretase | APH-1 (alpha and beta); Presenilin (Psen1); nicastrin |
| Related. | (Ncstn); PEN-2 |
| Disorders | |
| Others | Nos1; Parp1; Nat1; Nat2 |
| Prion-related | Prp |
| disorders | |
| ALS | SOD1; ALS2; STEX; FUS; TARDBP; VEGF |
| | (VEGF-a; VEGF-b; V EGF-c) |
| Drug addiction | Prkce (alcohol); Drd2; Drd4; ABAT (alcohol); |
| | GRIA2; Grm5; Grin1; Htr1b; Grin2a; Drd3; Pdyn; |
| | Gria1 (alcohol) |
| Autism | Mecp2; BZRAP1; MDGA2; Sema5A; Neurexin 1; |
| | Fragile X (FMR2 (AFF2); FXR1; FXR2; Mglur5) |
| Alzheimer's | E1; CHIP; UCH; UBB; Tau; LRP; PICALM; |
| Disease | Clusterin; PS1; SORL1; CR1; Vld1r; Uba1; Uba3; |
| | CHIP28 (Aqp1, Aquaporin 1); Uchl1; Uchl3; APP |
| Inflammation | 1L-10; IL-1 (1L-1a; IL-1b); 1L-13; IL-17 (IL-17a |
| | (CTLA8); IL-17b; IL-17c; IL-17d; IL-17f); II-23; |
| | Cx3er1; ptpn22; TNFa; NOD2/CARD15 for IBD; IL- |
| | 6; 1L-12 (1L-12a; 1L-12b); CTLA4; Cx3cl1 |
| Parkinson's | x-Synuclein; DJ-1; LRRK2; Parkin; PINK1 |
| Disease | |

TABLE B

| | |
|---|---|
| Blood and | Anemia (CDAN1, CDA1, RPS19, DBA, PKLR, |
| coagulation diseases | PK1, NT5C3, UMPH1, PSN1, RHAG, RH50A, |
| and disorders | NRAMP2, SPTB, ALAS2, ANH1, ASB, ABCB7, |
| | ABC7, ASAT); Bare lymphocyte syndrome |
| | (TAPBP, TPSN, TAP2, ABCB3, PSF2, RING11, |
| | MHC2TA, C2TA, RFX5, RFXAP, RFX5), |
| | Bleeding disorders (TBXA2R, P2RX1, P2X1); |
| | Factor H and factor H-like 1 (HF1, CFH, HUS); |
| | Factor V and factor VIII (MCFD2); Factor VII |
| | deficiency (F7); Factor X deficiency (F10); Factor |
| | XI deficiency (F11); Factor XII deficiency (F12, |
| | HAF); Factor XIIIA deficiency (F13A1, F13A); |
| | Factor XIIIB deficiency (F13B); Fanconi anemia |
| | (FANCA, FACA, FA1, FA, FAA, FAAP95, |
| | FAAP90, FLJ34064, FANCB, FANCC, FACC, |
| | BRCA2, FANCD1, FANCD2, FANCD, FACD, |
| | FAD, FANCE, FACE, FANCF, XRCC9, FANCG, |
| | BRIP1, BACH1, FANCJ, PHF9, FANCL, FANCM, |
| | KIAA1596); Hemophagocytic lymphohistiocytosis |
| | disorders (PRF1, HPLH2, UNC13D, MUNC13-4, |
| | HPLH3, HLH3, FHL3); Hemophilia A (F8, F8C, |
| | HEMA); Hemophilia B (F9, HEMB), Hemorrhagic |
| | disorders (PI, ATT, F5); Leukocyde deficiencies |
| | and disorders (ITGB2, CD18, LCAMB, LAD, |
| | EIF2B1, EIF2BA, EIF2B2, EIF2B3, EIF2B5, |
| | LVWM, CACH, CLE, EIF2B4); Sickle cell anemia |
| | (HBB); Thalassemia (HBA2, HBB, HBD, LCRB, |
| | HBA1). |
| Cell dysregulation | B-cell non-Hodgkin lymphoma (BCL7A, BCL7); |
| and oncology | Leukemia (TAL1 TCL5, SCL, TAL2, FLT3, NBS1, |
| diseases and | NBS, ZNFN1A1, IK1, LYF1, HOXD4, HOX4B, |
| disorders | BCR, CML, PHL, ALL, ARNT, KRAS2, RASK2, |
| | GMPS, AF10, ARHGEF12, LARG, KIAA0382, |
| | CALM, CLTH, CEBPA, CEBP, CHIC2, BTL, |
| | FLT3, KIT, PBT, LPP, NPM1, NUP214, D9S46E, |
| | CAN, CAIN, RUNX1, CBFA2, AML1, WHSC1L1, |
| | NSD3, FLT3, AF1Q, NPM1, NUMA1, ZNF145, |
| | PLZF, PML, MYL, STAT5B, AF10, CALM, |
| | CLTH, ARL11, ARLTS1, P2RX7, P2X7, BCR, |
| | CML, PHL, ALL, GRAF, NF1, VRNF, WSS, |
| | NFNS, PTPN11, PTP2C, SHP2, NS1, BCL2, |
| | CCND1, PRAD1, BCL1, TCRA, GATA1, GF1, |
| | ERYF1, NFE1, ABL1, NQO1, DIA4, NMOR1, |
| | NUP214, D9S46E, CAN, CAIN). |
| Inflammation and | AIDS (KIR3DL1, NKAT3, NKB1, AMB11, |
| immune related | KIR3DS1, IFNG, CXCL12, SDF1); Autoimmune |
| diseases and | lymphoproliferative syndrome (TNFRSF6, APT1, |
| disorders | FAS, CD95, ALPS1A); Combined immuno- |
| | deficiency, (IL2RG, SCIDX1, SCIDX, IMD4); |
| | HIV-1 (CCL5, SCYA5, D17S136E, TCP228), HIV |
| | susceptibility or infection (IL10, CSIF, CMKBR2, |
| | CCR2, CMKBR5, CCCKR5 (CCR5)); Immuno- |
| | deficiencies (CD3E, CD3G, AICDA, AID, HIGM2, |
| | TNFRSF5, CD40, UNG, DGU, HIGM4, TNFSF5, |
| | CD40LG, HIGM1, IGM, FOXP3, IPEX, AIID, |
| | XPID, PIDX, TNFRSF14B, TACI); Inflammation |
| | (IL-10, IL-1 (IL-1a, IL-1b), IL-13, IL-17 (IL-17a |
| | (CTLA8), IL-17b, IL-17c, IL-17d, IL-17f, II-23, |
| | Cx3cr1, ptpn22, TNFa, NOD2/CARD15 for IBD, |
| | IL-6, IL-12 (IL-12a, IL-12b), CTLA4, Cx3cl1); |
| | Severe combined immunodeficiencies (SCIDs) |
| | (JAK3, JAKL, DCLRE1C, ARTEMIS, SCIDA, |
| | RAG1, RAG2, ADA, PTPRC, CD45, LCA, IL7R, |
| | CD3D, T3D, IL2RG, SCIDX1, SCIDX, IMD4). |
| Metabolic, liver, | Amyloid neuropathy (TTR, PALB); Amyloidosis |
| kidney and protein | (APOA1, APP, AAA, CVAP, AD1, GSN, FGA, |
| diseases and | LYZ, TTR, PALB); Cirrhosis (KRT18, KRT8, |
| disorders | CIRH1A, NAIC, TEX292, KIAA1988); Cystic |
| | fibrosis (CFTR, ABCC7, CF, MRP7); Glycogen |
| | storage diseases (SLC2A2, GLUT2, G6PC, G6PT, |
| | G6PT1, GAA, LAMP2, LAMPB, AGL, GDE, |
| | GBE1, GYS2, PYGL, PFKM); Hepatic adenoma, |
| | 142330 (TCF1, HNF1A, MODY3), Hepatic failure, |
| | early onset, and neurologic disorder (SCOD1, |
| | SCO1), Hepatic lipase deficiency (LIPC), Hepato- |
| | blastoma, cancer and carcinomas (CTNNB1, |
| | PDGFRL, PDGRL, PRLTS, AXIN1, AXIN, |
| | CTNNB1, TP53, P53, LFS1, IGF2R, MPRI, MET, |
| | CASP8, MCH5; Medullary cystic kidney disease |
| | (UMOD, HNFJ, FJHN, MCKD2, ADMCKD2); |
| | Phenylketonuria (PAH, PKU1, QDPR, DHPR, |
| | PTS); Polycystic kidney and hepatic disease |
| | (FCYT, PKHD1, ARPKD, PKD1, PKD2, PKD4, |
| | PKDTS, PRKCSH, G19P1, PCLD, SEC63). |
| Muscular/Skeletal | Becker muscular dystrophy (DMD, BMD, MYF6), |
| diseases and | Duchenne Muscular Dystrophy (DMD, BMD); |
| disorders | Emery-Dreifuss muscular dystrophy (LMNA, |
| | LMN1, EMD2, FPLD, CMD1A, HGPS, LGMD1B, |
| | LMNA, LMN1, EMD2, FPLD, CMD1A); Facio- |
| | scapulohumeral muscular dystrophy (FSHMD1A, |
| | FSHD1A); Muscular dystrophy (FKRP, MDC1C, |
| | LGMD2I, LAMA2, LAMM, LARGE, KIAA0609, |
| | MDC1D, FCMD, TTID, MYOT, CAPN3, CANP3, |
| | DYSF, LGMD2B, SGCG, LGMD2C, DMDA1, |
| | SCG3, SGCA, ADL, DAG2, LGMD2D, DMDA2, |
| | SGCB, LGMD2E, SGCD, SGD, LGMD2F, |
| | CMD1L, TCAP, LGMD2G, CMD1N, TRIM32, |
| | HT2A, LGMD2H, FKRP, MDC1C, LGMD2I, |
| | TTN, CMD1G, TMD, LGMD2J, POMT1, CAV3, |
| | LGMD1C, SEPN1, SELN, RSMD1, PLEC1, |
| | PLTN, EBS1); Osteopetrosis (LRP5, BMND1, |
| | LRP7, LR3, OPPG, VBCH2, CLCN7, CLC7, |
| | OPTA2, OSTM1, GL, TCIRG1, TIRC7, OC116, |
| | OPTB1); Muscular atrophy (VAPB, VAPC, ALS8, |
| | SMN1, SMA1, SMA2, SMA3, SMA4, BSCL2, |
| | SPG17, GARS, SMAD1, CMT2D, HEXB, |
| | IGHMBP2, SMUBP2, CATF1, SMARD1). |
| Neurological and | ALS (SOD1, ALS2, STEX, FUS, TARDBP, VEGF |
| neuronal diseases | (VEGF-a, VEGF-b, VEGF-c); Alzheimer disease |
| and disorders | (APP, AAA, CVAP, AD1, APOE, AD2, PSEN2, |
| | AD4, STM2, APBB2, FE65L1, NOS3, PLAU, |
| | URK, ACE, DCP1, ACE1, MPO, PACIP1, |
| | PAXIP1L, PTIP, A2M, BLMH, BMH, PSEN1, |
| | AD3); Autism (Mecp2, BZRAP1, MDGA2, |
| | Sema5A, Neurexin 1, GLO1, MECP2, RTT, |
| | PPMX, MRX16, MRX79, NLGN3, NLGN4, |
| | KIAA1260, AUTSX2); Fragile X Syndrome |
| | (FMR2, FXR1, FXR2, mGLUR5); Huntington's |
| | disease and disease like disorders (HD, IT15, |
| | PRNP, PRIP, JPH3, JP3, HDL2, TBP, SCA17); |
| | Parkinson disease (NR4A2, NURR1, NOT, TINUR, |
| | SNCAIP, TBP, SCA17, SNCA, NACP, PARK1, |
| | PARK4, DJ1, PARK7, LRRK2, PARK8, PINK1, |
| | PARK6, UCHL1, PARK5, SNCA, NACP, PARK1, |
| | PARK4, PRKN, PARK2, PDJ, DBH, NDUFV2); |
| | Rett syndrome (MECP2, RTT, PPMX, MRX16, |
| | MRX79, CDKL5, STK9, MECP2, RTT, PPMX, |
| | MRX16, MRX79, x-Synuclein, DJ-1); Schizo- |
| | phrenia (Neuregulin1 (Nrg1), Erb4 (receptor for |
| | Neuregulin), Complexin1 (Cplx1), Tph1 Tryptophan |
| | hydroxylase, Tph2, Tryptophan hydroxylase 2, |
| | Neurexin 1, GSK3, GSK3a, GSK3b, 5-HTT |
| | (Slc6a4), COMT, DRD (Drd1a), SLC6A3, DAOA, |
| | DTNBP1, Dao (Dao1)); Secretase Related Disorders |
| | (APH-1 (alpha and beta), Presenilin (Psen1), |
| | nicastrin, (Ncstn), PEN-2, Nos1, Parp1, Nat1, |
| | Nat2); Trinucleotide Repeat Disorders (HTT |
| | (Huntington's Dx), SBMA/SMAX1/AR (Kennedy's |
| | Dx), FXN/X25 (Friedrich's Ataxia), ATX3 |
| | (Machado-Joseph's Dx), ATXN1 and ATXN2 |
| | (spinocerebellar ataxias), DMPK (myotonic |
| | dystrophy), Atrophin-1 and Atn1 (DRPLA Dx), |
| | CBP (Creb-BP - global instability), VLDLR |
| | (Alzheimer's), Atxn7, Atxn10). |
| Occular diseases | Age-related macular degeneration (Aber, Ccl2, Cc2, |
| and disorders | cp (ceruloplasmin), Timp3, cathepsinD, Vldlr, |
| | Ccr2); Cataract (CRYAA, CRYA1, CRYBB2, |
| | CRYB2, PITX3, BFSP2, CP49, CP47, CRYAA, |
| | CRYA1, PAX6, AN2, MGDA, CRYBA1, CRYB1, |
| | CRYGC, CRYG3, CCL, LIM2, MP19, CRYGD, |
| | CRYG4, BFSP2, CP49, CP47, HSF4, CTM, HSF4, |
| | CTM, MIP, AQP0, CRYAB, CRYA2, CTPP2, |
| | CRYBB1, CRYGD, CRYG4, CRYBB2, CRYB2, |
| | CRYGC, CRYG3, CCL, CRYAA, CRYA1, GJA8, |
| | CX50, CAE1, GJA3, CX46, CZP3, CAE3, CCM1, |
| | CAM, KRIT1); Corneal clouding and dystrophy |
| | (APOA1, TGFBI, CSD2, CDGG1, CSD, BIGH3, |
| | CDG2, TACSTD2, TROP2, M1S1, VSX1, RINX, |
| | PPCD, PPD, KTCN, COL8A2, FECD, PPCD2, |
| | PIP5K3, CFD); Cornea plana congenital (KERA, |
| | CNA2); Glaucoma (MYOC, TIGR, GLC1A, JOAG, |
| | GPOA, OPTN, GLC1E, FIP2, HYPL, NRP, |
| | CYP1B1, GLC3A, OPA1, NTG, NPG, CYP1B1, |
| | GLC3A); Leber congenital amaurosis (CRB1, |
| | RP12, CRX, CORD2, CRD, RPGRIP1, LCA6, |
| | CORD9, RPE65, RP20, AIPL1, LCA4, GUCY2D, |
| | GUC2D, LCA1, CORD6, RDH12, LCA3); |
| | Macular dystrophy (ELOVL4, ADMD, STGD2, |
| | STGD3, RDS, RP7, PRPH2, PRPH, AVMD, |
| | AOFMD, VMD2). |
| Epilepsy, | EPM2A, MELF, EPM2 |
| myoclonic, | |
| Lafora type, 254780 | |
| Epilepsy, | NHLRC1, EPM2A, EPM2B |
| myoclonic, | |
| Lafora type, 254780 | |
| Duchenne muscular | DMD, BMD |
| dystrophy, | |
| 310200 (3) | |
| AIDS, delayed/rapid | KIR3DL1, NKAT3, NKB1, AMB11, KIR3DS1 |
| progression to (3) | |
| AIDS, rapid | IFNG |
| progression to, | |
| 609423 (3) | |
| AIDS, resistance to | CXCL12, SDF1 |
| (3) | |
| Alpha 1-Antitrypsin | SERPINA1 [serpin peptidase inhibitor, clade A |
| Deficiency | (alpha-1 antiproteinase, antitrypsin), member 1]; |
| | SERPINA2 [serpin peptidase inhibitor, clade A |
| | (alpha-1 antiproteinase, antitrypsin), member 2]; |
| | SERPINA3 [serpin peptidase inhibitor, clade A |
| | (alpha-1 antiproteinase, antitrypsin), member 3]; |
| | SERPINA5 [serpin peptidase inhibitor, clade A |
| | (alpha-1 antiproteinase, antitrypsin), member 5]; |
| | SERPINA6 [serpin peptidase inhibitor, clade A |
| | (alpha-1 antiproteinase, antitrypsin), member 6]; |
| | SERPINA7 [serpin peptidase inhibitor, clade A |
| | (alpha-1 antiproteinase, antitrypsin), member 7];” |
| | AND “SERPLNA6 (serpin peptidase inhibitor, |
| | clade A (alpha-1 antiproteinase, antitrypsin), |
| | member 6) |

TABLE C

| CELLULAR | |
|---|---|
| FUNCTION | GENES |
| PI3K/AKT | PRKCE; ITGAM; ITGA5; IRAK1; PRKAA2; |
| Signaling | EIF2AK2; PTEN; EIF4E; PRKCZ; GRK6; |
| | MAPK1; TSC1; PLK1; AKT2; IKBKB; |
| | PIK3CA; CDK8; CDKN1B; NFKB2; BCL2; |
| | PIK3CB; PPP2R1A; MAPK8; BCL2L1; |
| | MAPK3; TSC2; ITGA1; KRAS; EIF4EBP1; |
| | RELA; PRKCD; NOS3; PRKAA1; MAPK9; |
| | CDK2; PPP2CA; PIM1; ITGB7; YWHAZ; ILK; |
| | TP53; RAF1; IKBKG; RELB; DYRK1A; |
| | CDKN1A; ITGB1; MAP2K2; JAK1; AKT1; |
| | JAK2; PIK3R1; CHUK; PDPK1; PPP2R5C; |
| | CTNNB1.; MAP2K1; NFKB1; PAK3; ITGB3; |
| | CCND1; GSK3A; FRAP1; SFN; ITGA2; TTK; |
| | CSNK1A1; BRAF; GSK3B; AKT3; FOXO1; |
| | SGK; HSP90AA1; RPS6KB1 |
| ERK/MAPK | PRKCE; ITGAM; ITGA5; HSPB1; IRAK1; |
| Signaling | PRKAA2; EIF2AK2; RAC1; RAP1A; TLN1; |
| | EIF4E; ELK1; GRK6; MAPK1; RAC2; PLK1; |
| | AKT2; PIK3CA; CDK8; CREB1; PRKCI; |
| | PTK2; FOS; RPS6KA4; PIK3CB; PPP2R1A; |
| | PIK3C3; MAPK8; MAPK3; ITGA1; ETS1; |
| | KRAS; MYCN; EIF4EBP1; PPARG; PRKCD; |
| | PRKAA1; MAPK9; SRC; CDK2; PPP2CA; |
| | PIM1; PIK3C2A; ITGB7; YWHAZ; PPP1CC; |
| | KSR1; PXN; RAF1; FYN; DYRK1A; ITGB1; |
| | MAP2K2; PAK4; PIK3R1; STAT3; PPP2R5C; |
| | MAP2K1; PAK3; ITGB3; ESR1; ITGA2; MYC; |
| | TTK; CSNK1A1; CRKL; BRAF; ATF4; |
| | PRKCA; SRF; STAT1; SGK |
| Glucocorticoid | RAC1; TAF4B; EP300; SMAD2; TRAF6; |
| Receptor Signaling | PCAF; ELK1; MAPK1; SMAD3; AKT2; |
| | IKBKB; NCOR2; UBE2I; PIK3CA; CREB1; |
| | FOS; HSPA5; NFKB2; BCL2; MAP3K14; |
| | STAT5B; PIK3CB; PIK3C3; MAPK8; BCL2L1; |
| | MAPK3; TSC22D3; MAPK10; NRIP1; KRAS; |
| | MAPK13; RELA; STAT5A; MAPK9; NOS2A; |
| | PBX1; NR3C1; PIK3C2A; CDKN1C; TRAF2; |
| | SERPINE1; NCOA3; MAPK14; TNF; RAF1; |
| | IKBKG; MAP3K7; CREBBP; CDKN1A; |
| | MAP2K2; JAK1; IL8; NCOA2; AKT1; JAK2; |
| | PIK3R1; CHUK; STAT3; MAP2K1; NFKB1; |
| | TGFBR1; ESR1; SMAD4; CEBPB; JUN; AR; |
| | AKT3; CCL2; MMP1; STAT1; IL6; HSP90AA1 |
| Axonal Guidance | PRKCE; ITGAM; ROCK1; ITGA5; CXCR4; |
| Signaling | ADAM12; IGF1; RAC1; RAP1A; E1F4E; |
| | PRKCZ; NRP1; NTRK2; ARHGEF7; SMO; |
| | ROCK2; MAPK1; PGF; RAC2; PTPN11; |
| | GNAS; AKT2; PIK3CA; ERBB2; PRKC1; |
| | PTK2; CFL1; GNAQ; PIK3CB; CXCL12; |
| | PIK3C3; WNT11; PRKD1; GNB2L1; ABL1; |
| | MAPK3; ITGA1; KRAS; RHOA; PRKCD; |
| | PIK3C2A; ITGB7; GLI2; PXN; VASP; RAF1; |
| | FYN; ITGB1; MAP2K2; PAK4; ADAM17; |
| | AKT1; PIK3R1; GLI1; WNT5A; ADAM10; |
| | MAP2K1; PAK3; ITGB3; CDC42; VEGFA; |
| | ITGA2; EPHA8; CRKL; RND1; GSK3B; |
| | AKT3; PRKCA |
| Ephrin Receptor | PRKCE; ITGAM; ROCK1; ITGA5; CXCR4; |
| Signaling | IRAK1; PRKAA2; EIF2AK2; RAC1; RAP1A; |
| | GRK6; ROCK2; MAPK1; PGF; RAC2; |
| | PTPN11; GNAS; PLK1; AKT2; DOK1; CDK8; |
| | CREB1; PTK2; CFL1; GNAQ; MAP3K14; |
| | CXCL12; MAPK8; GNB2L1; ABL1; MAPK3; |
| | ITGA1; KRAS; RHOA; PRKCD; PRKAA1; |
| | MAPK9; SRC; CDK2; PIM1; ITGB7; PXN; |
| | RAF1; FYN; DYRK1A; ITGB1; MAP2K2; |
| | PAK4, AKT1; JAK2; STAT3; ADAM10; |
| | MAP2K1; PAK3; ITGB3; CDC42; VEGFA; |
| | ITGA2; EPHA8; TTK; CSNK1A1; CRKL; |
| | BRAF; PTPN13; ATF4; AKT3; SGK |
| Actin Cytoskeleton | ACTN4; PRKCE; ITGAM; ROCK1; ITGA5; |
| Signaling | IRAK1; PRKAA2; EIF2AK2; RAC1; INS; |
| | ARHGEF7; GRK6; ROCK2; MAPK1; RAC2; |
| | PLK1; AKT2; PIK3CA; CDK8; PTK2; CFL1; |
| | PIK3CB; MYH9; DIAPH1; PIK3C3; MAPK8; |
| | F2R; MAPK3; SLC9A1; ITGA1; KRAS; RHOA; |
| | PRKCD; PRKAA1; MAPK9; CDK2; PIM1; |
| | PIK3C2A; ITGB7; PPP1CC; PXN; VIL2; RAF1; |
| | GSN; DYRK1A; ITGB1; MAP2K2; PAK4; |
| | PIP5K1A; PIK3R1; MAP2K1; PAK3; ITGB3; |
| | CDC42; APC; ITGA2; TTK; CSNK1A1; |
| | CRKL; BRAF; VAV3; SGK |
| Huntington's | PRKCE; IGF1; EP300; RCOR1.; PRKCZ; |
| Disease Signaling | HDAC4; TGM2; MAPK1; CAPNS1; AKT2; |
| | EGFR; NCOR2; SP1; CAPN2; PIK3CA; |
| | HDAC5; CREB1; PRKC1; HSPA5; REST; |
| | GNAQ; PIK3CB; PIK3C3; MAPK8; IGF1R; |
| | PRKD1; GNB2L1; BCL2L1; CAPN1; MAPK3; |
| | CASP8; HDAC2; HDAC7A; PRKCD; HDAC11; |
| | MAPK9; HDAC9; PIK3C2A; HDAC3; TP53; |
| | CASP9; CREBBP; AKT1; PIK3R1; PDPK1; |
| | CASP1; APAF1; FRAP1; CASP2; JUN; BAX; |
| | ATF4; AKT3; PRKCA; CLTC; SGK; HDAC6; |
| | CASP3 |
| Apoptosis | PRKCE; ROCK1; BID; IRAK1; PRKAA2; |
| Signaling | EIF2AK2; BAK1; BIRC4; GRK6; MAPK1; |
| | CAPNS1; PLK1; AKT2; IKBKB; CAPN2; |
| | CDK8; FAS; NFKB2; BCL2; MAP3K14; |
| | MAPK8; BCL2L1; CAPN1; MAPK3; CASP8; |
| | KRAS; RELA; PRKCD; PRKAA1; MAPK9; |
| | CDK2; PIM1; TP53; TNF; RAF1; IKBKG; |
| | RELB; CASP9; DYRK1A; MAP2K2; CHUK; |
| | APAF1; MAP2K1; NFKB1; PAK3; LMNA; |
| | CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; |
| Signaling | PTPN11; AKT2; IKBKB; PIK3CA; CREB1; |
| | SYK; NFKB2; CAMK2A; MAP3K14; PIK3CB; |
| | PIK3C3; MAPK8; BCL2L1; ABL1; MAPK3; |
| | ETS1; KRAS; MAPK13; RELA; PTPN6; |
| | MAPK9; EGR1; PIK3C2A; BTK; MAPK14; |
| | RAF1; IKBKG; RELB; MAP3K7; MAP2K2; |
| | AKT1; PIK3R1; CHUK; MAP2K1; NFKB1; |
| | CDC42; GSK3A; FRAP1; BCL6; BCL10; JUN; |
| | GSK3B; ATF4; AKT3; VAV3; RPS6KB1 |
| Leukocyte | ACTN4; CD44; PRKCE; ITGAM; ROCK1; |
| Extravasation | CXCR4; CYBA; RAC1; RAP1A; PRKCZ; |
| Signaling | ROCK2; RAC2; PTPN11; MMP14; PIK3CA; |
| | PRKCI; PTK2; PIK3CB; CXCL12; PIK3C3; |
| | MAPK8; PRKD1; ABL1; MAPK10; CYBB; |
| | MAPK13; RHOA; PRKCD; MAPK9; SRC; |
| | PIK3C2A; BTK; MAPK14; NOX1; PXN; VIL2; |
| | VASP; ITGB1; MAP2K2; CTNND1; PIK3R1; |
| | CTNNB1; CLDN1; CDC42; F11R; ITK; CRKL; |
| | VAV3; CTTN; PRKCA; MMP1; MMP9 |
| Integrin Signaling | ACTN4; ITGAM; ROCK1; ITGA5; RAC1; |
| | PTEN; RAP1A; TLN1; ARHGEF7; MAPK1; |
| | RAC2; CAPNS1; AKT2; CAPN2; P1K3CA; |
| | PTK2; PIK3CB; PIK3C3; MAPK8; CAV1; |
| | CAPN1; ABL1; MAPK3; ITGA1; KRAS; |
| | RHOA; SRC; PIK3C2A; ITGB7; PPP1CC; ILK; |
| | PXN; VASP; RAF1; FYN; ITGB1; MAP2K2; |
| | PAK4; AKT1; PIK3R1; TNK2; MAP2K1; |
| | PAK3; ITGB3; CDC42; RND3; ITGA2; CRKL; |
| | BRAF; GSK3B; AKT3 |
| Acute Phase | IRAK1; SOD2; MYD88; TRAF6; ELK1; |
| Response Signaling | MAPK1; PTPN11; AKT2; IKBKB; PIK3CA; |
| | FOS; NFKB2; MAP3K14; PIK3CB; MAPK8; |
| | RIPK1; MAPK3; IL6ST; KRAS; MAPK13; |
| | IL6R; RELA; SOCS1; MAPK9; FTL; NR3C1; |
| | TRAF2; SERPINE1; MAPK14; TNF; RAF1; |
| | PDK1; IKBKG; RELB; MAP3K7; MAP2K2; |
| | AKT1; JAK2; PIK3R1; CHUK; STAT3; |
| | MAP2K1; NFKB1; FRAP1; CEBPB; JUN; |
| | AKT3; IL1R1; IL6 |
| PTEN Signaling | ITGAM; ITGA5; RAC1; PTEN; PRKCZ; |
| | BCL2L11; MAPK1; RAC2; AKT2; EGFR; |
| | IKBKB; CBL; PIK3CA; CDKN1B; PTK2; |
| | NFKB2; BCL2; PIK3CB; BCL2L1; MAPK3; |
| | ITGA1; KRAS; ITGB7; ILK; PDGFRB; INSR; |
| | RAF1; IKBKG; CASP9; CDKN1A; ITGB1; |
| | MAP2K2; AKT1; PIK3R1; CHUK; PDGFRA; |
| | PDPK1; MAP2K1; NFKB1; ITGB3; CDC42; |
| | CCND1; GSK3A; ITGA2; GSK3B; AKT3; |
| | FOXO1; CASP3; RPS6KB1 |
| p53 Signaling | PTEN; EP300; BBC3; PCAF; FASN; BRCA1; |
| | GADD45A; BIRC5; AKT2; PIK3CA; CHEK1; |
| | TP53INP1; BCL2; PIK3CB; PIK3C3; MAPK8; |
| | THBS1; ATR; BCL2L1; E2F1; PMAIP1; |
| | CHEK2; TNFRSF10B; TP73; RB1; HDAC9; |
| | CDK2; PIK3C2A; MAPK14; TP53; LRDD; |
| | CDKN1A; HIPK2; AKT1; RIK3R1; RRM2B; |
| | APAF1; CTNNB1; SIRT1; CCND1; PRKDC; |
| | ATM; SFN; CDKN2A; JUN; SNAI2; GSK3B; |
| | BAX; AKT3 |
| Aryl Hydrocarbon | HSPB1; EP300; FASN; TGM2; RXRA; |
| Receptor | MAPK1; NQO1; NCOR2; SP1; ARNT; |
| Signaling | CDKN1B; FOS; CHEK1; SMARCA4; NFKB2; |
| | MAPK8; ALDH1A1; ATR; E2F1; MAPK3; |
| | NRIP1; CHEK2; RELA; TP73; GSTP1; RB1; |
| | SRC; CDK2; AHR; NFE2L2; NCOA3; TP53; |
| | TNF; CDKN1A; NCOA2; APAF1; NFKB1; |
| | CCND1; ATM; ESR1; CDKN2A; MYC; JUN; |
| | ESR2; BAX; IL6; CYP1B1; HSP90AA1 |
| Xenobiotic | PRKCE; EP300; PRKCZ; RXRA; MAPK1; |
| Metabolism | NQO1; NCOR2; PIK3CA; ARNT; PRKCI; |
| Signaling | NFKB2; CAMK2A; PIK3CB; PPP2R1A; |
| | PIK3C3; MAPK8; PRKD1; ALDH1A1; |
| | MAPK3; NRIP1; KRAS; MAPK13; PRKCD; |
| | GSTP1; MAPK9; NOS2A; ABCB1; AHR; |
| | PPP2CA; FTL; NFE2L2; PIK3C2A; |
| | PPARGC1A; MAPK14; TNF; RAF1; CREBBP; |
| | MAP2K2; PIK3R1; PPP2R5C; MAP2K1; |
| | NFKB1; KEAP1; PRKCA; EIF2AK3; IL6; |
| | CYP1B1; HSP90AA1 |
| SAPK/JNK | PRKCE; IRAK1; PRKAA2; EIF2AK2; RAC1; |
| Signaling | ELK1; GRK6; MAPK1; GADD45A; RAC2; |
| | PLK1; AKT2; PIK3CA; FADD; CDK8; |
| | PIK3CB; PIK3C3; MAPK8; RIPK1; GNB2L1; |
| | IRS1; MAPK3; MAPK10; DAXX; KRAS; |
| | PRKCD; PRKAA1; MAPK9; CDK2; PIM1; |
| | PIK3C2A; TRAF2; TP53; LCK; MAP3K7; |
| | DYRK1A; MAP2K2; PIK3R1; MAP2K1; |
| | PAK3; CDC42; JUN; TTK; CSNK1A1; CRKL; |
| | BRAF; SGK |
| PPAr/RXR | PRKAA2; EP300; INS; SMAD2; TRAF6; |
| Signaling | PPARA; FASN; RXRA; MAPK1; SMAD3; |
| | GNAS; IKBKB; NCOR2; ABCA1; GNAQ; |
| | NFKB2; MAP3K14; STAT5B; MAPK8; IRS1; |
| | MAPK3; KRAS; RELA; PRKAA1; |
| | PPARGC1A; NCOA3; MAPK14; INSR; RAF1; |
| | IKBKG; RELB; MAP3K7; CREBBP; MAP2K2; |
| | JAK2; CHUK; MAP2K1; NFKB1; TGFBR1; |
| | SMAD4; JUN; IL1R1; PRKCA; IL6; |
| | HSP90AA1; ADIPOQ |
| NF-KB Signaling | IRAK1; EIF2AK2; EP300; INS; MYD88; |
| | PRKCZ: TRAF6; TBK1; AKT2; EGFR; IKBKB; |
| | PIK3CA; BTRC; NFKB2; MAP3K14; PIK3CB; |
| | PIK3C3; MAPK8; RIPK1; HDAC2; KRAS; |
| | RELA; PIK3C2A; TRAF2; TLR4: PDGFRB; |
| | TNF; INSR; LCK; IKBKG; RELB; MAP3K7; |
| | CREBBP; AKT1; PIK3R1; CHUK; PDGFRA; |
| | NFKB1; TLR2; BCL10; GSK3B; AKT3; |
| | TNFAIP3; IL1R1 |
| Neuregulin | ERBB4; PRKCE; ITGAM; ITGA5: PTEN; |
| Signaling | PRKCZ; ELK1; MAPK1; PTPN11; AKT2; |
| | EGFR; ERBB2; PRKCI; CDKN1B; STAT5B; |
| | PRKD1; MAPK3; ITGA1; KRAS; PRKCD; |
| | STAT5A; SRC; ITGB7; RAF1; ITGB1; |
| | MAP2K2; ADAM17; AKT1; PIK3R1; PDPK1; |
| | MAP2K1; ITGB3; EREG; FRAP1; PSEN1; |
| | ITGA2; MYC; NRG1; CRKL; AKT3; PRKCA; |
| | HSP90AA1; RPS6KB1 |
| Wnt & Beta catenin | CD44; EP300; LRP6; DVL3; CSNK1E; GJA1; |
| Signaling | SMO; AKT2; PIN1; CDH1; BTRC; GNAQ; |
| | MARK2; PPP2R1A; WNT11; SRC; DKK1; |
| | PPP2CA; SOX6; SFRP2: ILK; LEF1; SOX9; |
| | TP53; MAP3K7; CREBBP; TCF7L2; AKT1; |
| | PPP2R5C; WNT5A; LRP5; CTNNB1; TGFBR1; |
| | CCND1; GSK3A; DVL1; APC; CDKN2A; |
| | MYC; CSNK1A1; GSK3B; AKT3; SOX2 |
| Insulin Receptor | PTEN; INS; EIF4E; PTPN1; PRKCZ; MAPK1; |
| Signaling | TSC1; PTPN11; AKT2; CBL; PIK3CA; PRKCI; |
| | PIK3CB; PIK3C3; MAPK8; IRS1; MAPK3; |
| | TSC2; KRAS; EIF4EBP1; SLC2A4; PIK3C2A; |
| | PPP1CC; INSR; RAF1; FYN; MAP2K2; JAK1; |
| | AKT1; JAK2; PIK3R1; PDPK1; MAP2K1; |
| | GSK3A; FRAP1; CRKL; GSK3B; AKT3; |
| | FOXO1; SGK; RPS6KB1 |
| IL-6 Signaling | HSPB1; TRAF6; MAPKAPK2; ELK1; MAPK1; |
| | PTPN11; IKBKB; FOS; NFKB2: MAP3K14; |
| | MAPK8; MAPK3; MAPK10; IL6ST; KRAS; |
| | MAPK13; IL6R; RELA; SOCS1; MAPK9; |
| | ABCB1; TRAF2; MAPK14; TNF; RAF1; |
| | IKBKG; RELB; MAP3K7; MAP2K2; IL8; |
| | JAK2; CHUK; STAT3; MAP2K1; NFKB1; |
| | CEBPB; JUN; IL1R1; SRF; IL6 |
| Hepatic Cholestasis | PRKCE; IRAK1; INS; MYD88; PRKCZ; |
| | TRAF6; PPARA; RXRA; IKBKB; PRKCI; |
| | NFKB2; MAP3K14; MAPK8; PRKD1; |
| | MAPK10; RELA; PRKCD; MAPK9; ABCB1; |
| | TRAF2; TLR4; TNF; INSR; IKBKG; RELB; |
| | MAP3K7; IL8; CHUK; NR1H2; TJP2; NFKB1; |
| | ESR1; SREBF1; FGFR4; JUN; IL1R1; PRKCA; |
| | IL6 |
| IGF-1 Signaling | IGF1; PRKCZ; ELK1; MAPK1; PTPN11; |
| | NEDD4; AKT2; PIK3CA; PRKC1; PTK2; FOS; |
| | PIK3CB; PIK3C3; MAPK8; 1GF1R; IRS1; |
| | MAPK3; IGFBP7; KRAS; PIK3C2A; YWHAZ; |
| | PXN; RAF1; CASP9; MAP2K2; AKT1; |
| | PIK3R1; PDPK1; MAP2K1; IGFBP2; SFN; |
| | JUN; CYR61; AKT3; FOXO1; SRF; CTGF; |
| | RPS6KB1 |
| NRF2-mediated | PRKCE; EP300; SOD2; PRKCZ; MAPK1; |
| Oxidative | SQSTM1; NQO1; PIK3CA; PRKC1; FOS; |
| Stress Response | PIK3CB; P1K3C3; MAPK8; PRKD1; MAPK3; |
| | KRAS; PRKCD; GSTP1; MAPK9; FTL; |
| | NFE2L2; PIK3C2A; MAPK14; RAF1; |
| | MAP3K7; CREBBP; MAP2K2; AKT1; PIK3R1; |
| | MAP2K1; PPIB; JUN; KEAP1; GSK3B; ATF4; |
| | PRKCA; EIF2AK3; HSP90AA1 |
| Hepatic, Fibrosis/ | EDN1; IGF1; KDR; FLT1; SMAD2; FGFR1; |
| Hepatic Stellate | MET; PGF; SMAD3; EGFR; FAS; CSF1; |
| Cell Activation | NFKB2; BCL2; MYH9; IGF1R; IL6R; RELA; |
| | TLR4; PDGFRB; TNF; RELB; IL8; PDGFRA; |
| | NFKB1; TGFBR1; SMAD4; VEGFA; BAX; |
| | IL1R1; CCL2; HGF; MMP1; STAT1; IL6; |
| | CTGF; MMP9 |
| PPAR Signaling | EP300; INS; TRAF6; PPARA; RXRA; MAPK1; |
| | IKBKB; NCOR2; FOS; NFKB2; MAP3K14; |
| | STAT5B; MAPK3; NRIP1; KRAS; PPARG; |
| | RELA; STAT5A; TRAF2; PPARGC1A; |
| | PDGFRB; TNF; INSR; RAF1; IKBKG; RELB; |
| | MAP3K7; CREBBP; MAP2K2; CHUK; |
| | PDGFRA; MAP2K1; NFKB1; JUN; IL1R1; |
| | HSP90AA1 |
| Fc Epsilon RI | PRKCE; RAC1; PRKCZ; LYN; MAPK1; RAC2; |
| Signaling | PTPN11; AKT2; PIK3CA; SYK; PRKCI; |
| | PIK3CB; PIK3C3; MAPK8; PRKD1; MAPK3; |
| | MAPK10; KRAS; MAPK13; PRKCD; MAPK9; |
| | PIK3C2A; BTK; MAPK14; TNF; RAF1; FYN; |
| | MAP2K2; AKT1; PIK3R1; PDPK1; MAP2K1; |
| | AKT3; VAV3; PRKCA |
| G-Protein Coupled | PRKCE; RAP1A; RGS16; MAPK1; GNAS; |
| Receptor Signaling | AKT2; IKBKB; PIK3CA; CREB1; GNAQ; |
| | NFKB2; CAMK2A; PIK3CB; PIK3C3; MAPK3; |
| | KRAS; RELA; SRC; PIK3C2A; RAF1; IKBKG; |
| | RELB; FYN; MAP2K2; AKT1; PIK3R1; |
| | CHUK; PDPK1; STAT3; MAP2K1; NFKB1; |
| | BRAF; ATF4; AKT3; PRKCA |
| Inositol Phosphate | PRKCE; IRAK1; PRKAA2; EIF2AK2; PTEN; |
| Metabolism | GRK6; MAPK1; PLK1; AKT2; PIK3CA; CDK8; |
| | PIK3CB; PIK3C3; MAPK8; MAPK3; PRKCD; |
| | PRKAA1; MAPK9; CDK2; PIM1; PIK3C2A; |
| | DYRK1A; MAP2K2; PIP5K1A; PIK3R1; |
| | MAP2K1; PAK3; ATM; TTK; CSNK1A1; |
| | BRAF; SGK |
| PDGF Signaling | EIF2AK2; ELK1; ABL2; MAPK1; PIK3CA; |
| | FOS; PIK3CB; PIK3C3; MAPK8; CAV1; ABL1; |
| | MAPK3; KRAS; SRC; PIK3C2A; PDGFRB; |
| | RAF1; MAP2K2; JAK1; JAK2; PIK3R1; |
| | PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; |
| | MAPK1; PGF; AKT2; PIK3CA; ARNT; PTK2; |
| | BCL2; PIK3CB; PIK3C3; BCL2L1; MAPK3; |
| | KRAS; HIF1A; NOS3; PIK3C2A; PXN; RAF1; |
| | MAP2K2; ELAVL1; AKT1; PIK3R1; MAP2K1; |
| | SFN; VEGFA; AKT3; FOXO1; PRKCA |
| Natural Killer Cell | PRKCE; RAC1; PRKCZ; MAPK1; RAC2; |
| Signaling | PTPN11; KIR2DL3; AKT2; PIK3CA; SYK; |
| | PRKCI; PIK3CB; PIK3C3; PRKD1; MAPK3; |
| | KRAS; PRKCD; PTPN6; PIK3C2A; LCK; |
| | RAF1; FYN; MAP2K2; PAK4; AKT1; PIK3R1; |
| | MAP2K1; PAK3; AKT3; VAV3; PRKCA |
| Cell Cycle: G1/S | HDAC4; SMAD3; SUV39H1; HDAC5; |
| Checkpoint | CDKN1B; BTRC; ATR; ABL1; E2F1; HDAC2; |
| Regulation | HDAC7A; RB1; HDAC11; HDAC9; CDK2; |
| | E2F2; HDAC3; TP53; CDKN1A; CCND1; |
| | E2F4; ATM; RBL2; SMAD4; CDKN2A; MYC; |
| | NRG1; GSK3B; RBL1; HDAC6 |
| T Cell Receptor | RAC1; ELK1; MAPK1; IKBKB; CBL; PIK3CA; |
| Signaling | FOS; NFKB2; PIK3CB; PIK3C3; MAPK8; |
| | MAPK3; KRAS; RELA, PIK3C2A; BTK; LCK; |
| | RAF1; IKBKG; RELB, FYN; MAP2K2; |
| | PIK3R1; CHUK; MAP2K1; NFKB1; ITK; |
| | BCL10; JUN; VAV3 |
| Death Receptor | CRADD; HSPB1; BID; BIRC4; TBK1; IKBKB; |
| Signaling | FADD; FAS; NFKB2; BCL2; MAP3K14; |
| | MAPK8; RIPK1; CASP8; DAXX; TNFRSF10B; |
| | RELA; TRAF2; TNF; IKBKG; RELB; CASP9; |
| | CHUK; APAF1; NFKB1; CASP2; BIRC2; |
| | CASP3; BIRC3 |
| FGF Signaling | RAC1; FGFR1; MET; MAPKAPK2; MAPK1; |
| | PTPN11; AKT2; PIK3CA; CREB1; PIK3CB; |
| | PIK3C3; MAPK8; MAPK3; MAPK13; PTPN6; |
| | PIK3C2A; MAPK14; RAF1; AKT1; PIK3R1; |
| | STAT3; MAP2K1; FGFR4; CRKL; ATF4; |
| | AKT3; PRKCA; HGF |
| GM-CSF Signaling | LYN; ELK1; MAPK1; PTPN11; AKT2; |
| | PIK3CA; CAMK2A; STAT5B; PIK3CB; |
| | PIK3C3; GNB2L1; BCL2L1; MAPK3; ETS1; |
| | KRAS; RUNX1; PIM1; PIK3C2A; RAF1; |
| | MAP2K2; AKT1; JAK2; PIK3R1; STAT3; |
| | MAP2K1; CCND1; AKT3; STAT1 |
| Amyotrophic | BID; IGF1; RAC1; BIRC4; PGF; CAPNS1; |
| Lateral Sclerosis | CAPN2; PIK3CA; BCL2; PIK3CB; PIK3C3; |
| Signaling | BCL2L1; CAPN1; PIK3C2A; TP53; CASP9; |
| | PIK3R1; RAB5A; CASP1; APAF1; VEGFA; |
| | BIRC2; BAX; AKT3; CASP3; BIRC3 |
| JAK/Stat Signaling | PTPN1; MAPK1; PTPN11; AKT2; PIK3CA; |
| | STAT5B; PIK3CB; PIK3C3; MAPK3; KRAS; |
| | SOCS1; STAT5A; PTPN6; PIK3C2A; RAF1; |
| | CDKN1A; MAP2K2; JAK1; AKT1; JAK2; |
| | PIK3R1; STAT3; MAP2K1; FRAP1; AKT3; |
| | STAT1 |
| Nicotinate and | PRKCE; IRAK1; PRKAA2; EIF2AK2; GRK6; |
| Nicotinamide | MAPK1; PLK1; AKT2; CDK8; MAPK8; |
| Metabolism | MAPK3; PRKCD; PRKAA1; PBEF1; MAPK9; |
| | CDK2; PIM1; DYRK1A; MAP2K2; MAP2K1; |
| | PAK3; NT5E; TTK; CSNK1A1; BRAF; SGK |
| Chemokine | CXCR4; ROCK2; MAPK1; PTK2; FOS; CFL1; |
| Signaling | GNAQ; CAMK2A; CXCL12; MAPK8; MAPK3; |
| | KRAS; MAPK13; RHOA; CCR3; SRC; |
| | PPP1CC; MAPK14; NOX1; RAF1; MAP2K2; |
| | MAP2K1; JUN; CCL2; PRKCA |
| IL-2 Signaling | ELK1; MAPK1; PTPN11; AKT2; PIK3CA; |
| | SYK; FOS; STAT5B; PIK3CB; PIK3C3; |
| | MAPK8; MAPK3; KRAS; SOCS1; STAT5A; |
| | PIK3C2A: LCK; RAF1; MAP2K2; JAK1; |
| | AKT1; PIK3R1; MAP2K1; JUN; AKT3 |
| Synaptic Long | PRKCE; IGF1; PRKCZ; PRDX6; LYN; |
| Term Depression | MAPK1; GNAS; PRKC1; GNAQ; PPP2R1A; |
| | IGF1R; PRKID1; MAPK3; KRAS; GRN; |
| | PRKCD; NOS3; NOS2A; PPP2CA; YWHAZ; |
| | RAF1; MAP2K2; PPP2R5C; MAP2K1; PRKCA |
| Estrogen Receptor | TAF4B; EP300; CARM1; PCAF; MAPK1; |
| Signaling | NCOR2; SMARCA4; MAPK3; NRIP1; KRAS; |
| | SRC; NR3C1; HDAC3; PPARGC1A; RBM9; |
| | NCOA3; RAF1; CREBBP; MAP2K2; NCOA2; |
| | MAP2K1; PRKDC; ESR1; ESR2 |
| Protein | TRAF6; SMURF1; BIRC4; BRCA1; UCHL1; |
| Ubiquitination | NEDD4; CBL; UBE2I; BTRC; HSPA5; USP7; |
| Pathway | USP10; FBXW7; USP9X; STUB1; USP22; |
| | B2M; BIRC2; PARK2; USP8; USP1; VHL; |
| | HSP90AA1; BIRC3 |
| IL-10 Signaling | TRAF6; CCR1; ELK1; IKBKB; SP1; FOS; |
| | NFKB2; MAP3K14; MAPK8; MAPK13; RELA; |
| | MAPK14; TNF; IKBKG; RELB; MAP3K7; |
| | JAK1; CHUK; STAT3; NFKB1; JUN; IL1R1; |
| | IL6 |
| VDR/RXR | PRKCE; EP300; PRKCZ; RXRA; GADD45A; |
| Activation | HES1; NCOR2; SP1; PRKC1; CDKN1B; |
| | PRKD1; PRKCD; RUNX2; KLF4; YY1; |
| | NCOA3; CDKN1A; NCOA2; SPP1; LRP5; |
| | CEBPB; FOXO1; PRKCA |
| TGF-beta Signaling | EP300; SMAD2; SMURF1; MAPK1; SMAD3; |
| | SMAD1; FOS; MAPK8; MAPK3; KRAS; |
| | MAPK9; RUNX2; SERPINE1; RAF1; |
| | MAP3K7; CREBBP; MAP2K2; MAP2K1; |
| | TGFBR1; SMAD4; JUN; SMAD5 |
| Toll-like Receptor | IRAK1; EIF2AK2; MYD88; TRAF6; PPARA; |
| Signaling | ELK1; IKBKB; FOS; NFKB2; MAP3K14; |
| | MAPK8; MAPK13; RELA; TLR4; MAPK14; |
| | IKBKG; RELB; MAP3K7; CHUK; NFKB1; |
| | TLR2; JUN |
| p38 MAPK | HSPB1; IRAK1; TRAF6; MAPKAPK2; ELK1; |
| Signaling | FADD; FAS; CREB1; DDIT3; RPS6KA4; |
| | DAXX; MAPK13; TRAF2; MAPK14; TNF; |
| | MAP3K7; TGFBR1; MYC; ATF4; IL1R1; SRF; |
| | STAT1 |
| Neurotrophin/TRK | NTRK2; MAPK1; PTPN11; PIK3CA; CREB1; |
| Signaling | FOS; PIK3CB; PIK3C3; MAPK8; MAPK3; |
| | KRAS; PIK3C2A; RAF1; MAP2K2; AKT1; |
| | PIK3R1; PDPK1; MAP2K1; CDC42; JUN; |
| | ATF4 |
| FXR/RXR | INS; PPARA; FASN; RXRA; AKT2; SDC1; |
| Activation | MAPK8; APOB; MAPK10; PPARG; MTTP; |
| | MAPK9; PPARGC1A; TNF; CREBBP; AKT1; |
| | SREBF1; FGFR4; AKT3; FOXO1 |
| Synaptic Long | PRKCE; RAP1A; EP300; PRKCZ; MAPK1; |
| Term Potentiation | CREB1; PRKC1; GNAQ; CAMK2A; PRKD1; |
| | MAPK3; KRAS; PRKCD; PPP1CC; RAF1; |
| | CREBBP; MAP2K2; MAP2K1; ATF4; PRKCA |
| Calcium Signaling | RAP1A; EP300; HDAC4; MAPK1; HDAC5; |
| | CREB1; CAMK2A; MYH9; MAPK3; HDAC2; |
| | HDAC7A; HDAC11; HDAC9; HDAC3; |
| | CREBBP; CALR; CAMKK2; ATF4; HDAC6 |
| EGF Signaling | ELK1; MAPK1; EGFR; PIK3CA; FOS; |
| | PIK3CB; PIK3C3; MAPK8; MAPK3; PIK3C2A; |
| | RAF1; JAK1; PIK3R1; STAT3; MAP2K1; JUN; |
| | PRKCA; SRF; STAT1 |
| Hypoxia Signaling | EDN1; PTEN; EP300; NQO1; UBE21; CREB1; |
| in the | ARNT; HIF1A; SLC2A4; NOS3; TP53; LDHA; |
| Cardiovascular | AKT1; ATM; VEGFA; JUN; ATF4; VHL; |
| System | HSP90AA1 |
| LPS/IL-1 Mediated | IRAK1; MYD88; TRAF6; PPARA; RXRA; |
| Inhibition | ABCA1, MAPK8; ALDH1A1; GSTP1; MAPK9; |
| of RXR Function | ABCB1; TRAF2; TLR4; TNF; MAP3K7; |
| | NR1H2; SREBF1; JUN; IL1R1 |
| LXR/RXR | FASN; RXRA; NCOR2; ABCA1; NFKB2; |
| Activation | IRF3; RELA; NOS2A; TLR4; TNF; RELB; |
| | LDLR; NR1H2; NFKB1; SREBF1; IL1R1; |
| | CCL2; IL6; MMP9 |
| Amyloid | PRKCE; CSNK1E; MAPK1; CAPNS1; AKT2; |
| Processing | CAPN2; CAPN1; MAPK3; MAPK13; MAPT; |
| | MAPK14; AKT1; PSEN1; CSNK1A1; GSK3B; |
| | AKT3; APP |
| IL-4 Signaling | AKT2; PIK3CA; PIK3CB; PIK3C3; IRS1; |
| | KRAS; SOCS1; PTPN6; NR3C1; PIK3C2A; |
| | JAK1; AKT1; JAK2; PIK3R1; FRAP1; AKT3; |
| | RPS6KB1 |
| Cell Cycle: G2/M | EP300; PCAF; BRCA1; GADD45A; PLK1; |
| DNA Damage | BTRC; CHEK1; ATR; CHEK2; YWHAZ; TP53; |
| Checkpoint | CDKN1A; PRKDC; ATM; SFN; CDKN2A |
| Regulation | |
| Nitric Oxide | KDR; FLT1; PGF; AKT2; PIK3CA; PIK3CB; |
| Signaling in the | PIK3C3; CAV1; PRKCD; NOS3; PIK3C2A; |
| Cardiovascular | AKT1; PIK3R1; VEGFA; AKT3; HSP90AA1 |
| System | |
| Purine Metabolism | NME2; SMARCA4; MYH9; RRM2; ADAR; |
| | EIF2AK4; PKM2; ENTPD1; RAD51; RRM2B; |
| | TJP2; RAD51C; NT5E; POLD1; NME1 |
| cAMP-mediated | RAP1A; MAPK1; GNAS; CREB1; CAMK2A; |
| Signaling | MAPK3; SRC; RAF1; MAP2K2; STAT3; |
| | MAP2K1; BRAF; ATF4 |
| Mitochondrial | SOD2; MAPK8; CASP8; MAPK10; MAPK9; |
| Dysfunction | CASP9; PARK7; PSEN1; PARK2; APP; CASP3 |
| Notch Signaling | HES1; JAG1; NUMB; NOTCH4; ADAM17; |
| | NOTCH2; PSEN1; NOTCH3; NOTCH1; DLL4 |
| Endoplasmic | HSPA5; MAPK8; XBP1; TRAF2; ATF6; |
| Reticulum Stress | CASP9; ATF4; EIF2AK3; CASP3 |
| Pathway | |
| Pyrimidine | NME2; AICDA; RRM2; EIF2AK4; ENTPD1; |
| Metabolism | RRM2B; NT5E; POLD1; NME1 |
| Parkinson's | UCHL1; MAPK8; MAPK13; MAPK14; CASP9; |
| Signaling | PARK7; PARK2; CASP3 |
| Cardiac & Beta | GNAS; GNAQ; PPP2R1A; GNB2L1; PPP2CA; |
| Adrenergic | PPP1CC; PPP2R5C |
| Signaling | |
| Glycolysis/ | HK2; GCK; GPI; ALDH1A1; PKM2; LDHA; |
| Gluconeogenesis | HK1 |
| Interferon Signaling | IRF1; SOCS1; JAK1; JAK2; IFITM1; STAT1; |
| | IFIT3 |
| Sonic Hedgehog | ARRB2; SMO; GLI2; DYRK1A; GLI1; GSK3B; |
| Signaling | DYRKIB |
| Glycero- | PLD1; GRN; GPAM; YWHAZ; SPHK1; SPHK2 |
| phospholipid | |
| Metabolism | |
| Phospholipid | PRDX6; PLD1; GRN; YWHAZ; SPHK1; |
| Degradation | SPHK2 |
| Tryptophan | SIAH2; PRMT5; NEDD4; ALDH1A1; CYP1B1; |
| Metabolism | SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine | DNMT1; DNMT3B; AHCY; DNMT3A |
| Metabolism | |
| Pyruvate | GLO1; ALDH1A1; PKM2; LDHA |
| Metabolism | |
| Arginine and | ALDH1A1; NOS3; NOS2A |
| Proline | |
| Metabolism | |
| Eicosanoid | PRDX6; GRN; YWHAZ |
| Signaling | |
| Fructose and | HK2; GCK; HK1 |
| Mannose | |
| Metabolism | |
| Galactose | HK2; GCK; HK1 |
| Metabolism | |
| Stilbene, Coumarine | PRDX6; PRDX1; TYR |
| and Lignin | |
| Biosynthesis | |
| Antigen | CALR; B2M |
| Presentation | |
| Pathway | |
| Biosynthesis of | NQO1; DHCR7 |
| Steroids | |
| Butanoate | ALDH1A1; NLGN1 |
| Metabolism | |
| Citrate Cycle | IDH2; IDH1 |
| Fatty Acid | ALDH1A1; CYP1B1 |
| Metabolism | |
| Glycero- | PRDX6; CHKA |
| phospholipid | |
| Metabolism | |
| Histidine | PRMT5; ALDH1A1 |
| Metabolism | |
| Inositol Metabolism | ERO1L; APEX1 |
| Metabolism of | GSTP1; CYP1B1 |
| Xenobiotics by | |
| Cytochrome p450 | |
| Methane | PRDX6; PRDX1 |
| Metabolism | |
| Phenylalanine | PRDX6; PRDX1 |
| Metabolism | |
| Propanoate | ALDH1A1; LDHA |
| Metabolism | |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and | PRMT5 |
| Estrogen | |
| Metabolism | |
| Ascorbate and | ALDH1A1 |
| Aldarate | |
| Metabolism | |
| Bile Acid | ALDH1A1 |
| Biosynthesis | |
| Cysteine | LDHA |
| Metabolism | |
| Fatty Acid | FASN |
| Biosynthesis | |
| Glutamate Receptor | GNB2L1 |
| Signaling | |
| NRF2-mediated | PRDX1 |
| Oxidative | |
| Stress Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin | TYR |
| Metabolism | |
| Tyrosine | PRMT5, TYR |
| Metabolism | |
| Ubiquinone | PRMT5 |
| Biosynthesis | |
| Valine, Leucine and | ALDH1A1 |
| Isoleucine | |
| Degradation | |
| Glycine, Serine and | CHKA |
| Threonine | |
| Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; |
| | Grk2; Trpa1; Pomc; Cgrp; Crf; Pka; Era; Nr2b; |
| | TRPM5; Prkaca; Prkacb; Prkar1a; Prkar2a |
| Mitochondrial | AIF; CytC; SMAC (Diablo); Aifm-1; Aifm-2 |
| Function | |
| Developmental | BMP-4; Chordin (Chrd); Noggin (Nog); WNT |
| Neurology | (Wnt2; Wnt2b; Wnt3a; Wnt4; Wnt5a; Wnt6; |
| | Wnt7b; Wnt8b; Wnt9a; Wnt9b; Wnt10a; |
| | Wnt10b; Wnt16); beta-catenin; Dkk-1; Frizzled |
| | related proteins; Otx-2; Gbx2; FGF-8; Reelin; |
| | Dab1; unc-86 (Pou4f1 or Brn3a); Numb; Reln |

What is claimed is:

1. A method of cleaving a target site of cellular DNA, the method comprising the steps of introducing an artificial chimeric molecule into a cell, wherein said artificial chimeric molecule comprises: a) a nucleic acid analogue, wherein said nucleic acid analogue has been designed to specifically bind to said target site of cellular DNA; and b) a nuclease domain, wherein said nuclease domain contains bleomycin or its derivatives, whereby said artificial chimeric molecule binds to said target site of cellular DNA and said target site of cellular DNA is cleaved by said nuclease domain.

2. The method of claim 1, wherein the nucleic acid analogue is peptide nucleic acid or gamma peptide nucleic acid.

3. The method of claim 1, wherein the bleomycin or its derivatives is selected from the group consisting of bleomycin A2, bleomycin B2, bleomycin A6, bleomycin A5, phleomycin, chloridenated and their deglycosylated derivatives.

4. A method of claim 1 can be used in killing a microorganism comprising contacting said organism with said artificial chimeric molecule, wherein said artificial chimeric molecule binds specifically to a target polynucleotide sequence of said microorganism.

5. A method according to claim 4 wherein the microorganism is selected from the group consisting of viruses, bacteria, and eukaryotic parasites.

6. A method of cleaving a target site of cellular DNA, the method comprising the steps of introducing an artificial chimeric molecule into a cell, wherein said artificial chimeric molecule comprises: a) a peptide nucleic acid, wherein said nucleic acid analogue has been designed to specifically bind to said target site DNA and comprises one or more pseudoisocytosine monomers; and b) a nuclease domain, wherein said nuclease domain contains bleomycin or its derivatives, whereby said artificial chimeric molecule binds to said target site of DNA and said target site of DNA is cleaved by said nuclease domain.

7. Said artificial chimeric molecule in claim 6 comprise Nuclear localization sequence

8. Said artificial chimeric molecule in claim 6 comprise Tat peptide


SEQUENCE LISTING (1 sequence)

SEQ ID NO: 1 - DNA, 25 bp, ARTIFICIAL SEQUENCE
  synthetic peptide nucleic acid
  MISC_FEATURE (1)..(1): linked to Lys-Lys-Lys
  MISC_FEATURE (1)..(1): pseudoisocytosine
  MISC_FEATURE (3)..(3): pseudoisocytosine
  MISC_FEATURE (6)..(6): pseudoisocytosine
  MISC_FEATURE (9)..(9): pseudoisocytosine
  MISC_FEATURE (10)..(11): linkedin by three  8-amino-2,6-dioxaoctanoic acid molecules
  MISC_FEATURE (25)..(25): linked to Lys-Lys-Lys
ntnttnttnttcttcttctcatttc